(19) United States
(12) Patent Application Publication
(10) Pub. No.: US 2009/0275630 A1
(43) Pub. Date: Nov. 5, 2009

(54) COSMETIC COMPOSITION COMPRISING AN IMIDOPERCARBOXYLIC ACID DERIVATIVE AND A N-ACYLATED AMINO ACID ESTER

(75) Inventors: Roxane Provost, Paris (FR); Geoffroy Remaut, Fresnes (FR); Cecile Kermorvan, Saint Maur Des Fosses (FR); Celine Moussay, Vanves (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/407,858

(22) Filed: Mar. 20, 2009

Related U.S. Application Data
(60) Provisional application No. 61/042,297, filed on Apr. 4, 2008.

(30) Foreign Application Priority Data
Mar. 28, 2008 (FR) 08 52018

(51) Int. Cl. A61K 31/403; A61K 8/37; A61K 8/49; A61Q 19/00; A61K 31/21; A61K 31/40; A61K 31/4035; A61P 17/00

(52) U.S. Cl. 514/411; 514/551; 514/425; 514/416

(57) ABSTRACT

Composition containing, in a physiologically acceptable medium, at least one imidopercarboxylic acid compound and at least one N-acylated amino acid ester of formula (A) as described herein.

### REFERENCE TO PRIOR APPLICATIONS

This application claims priority to U.S. provisional application 61/042,297, filed Apr. 4, 2008; and to French patent application 08 52018 filed Mar. 28, 2008, both incorporated herein by reference.

### FIELD OF THE INVENTION

The present invention relates to a composition, in particular a cosmetic or dermatological composition, comprising an imidopercarboxylic acid derivative (sometimes referred to herein as an imidopercarboxylic acid compound) and an N-acylated amino acid ester.

### BACKGROUND OF THE INVENTION

It is known to use imidopercarboxylic acid derivatives in topical compositions, in particular cosmetic or dermatological topical compositions, for example as antimicrobial or antifungal agents. Their activity in antiacne or lightening compositions is also known.

However, the use of these derivatives presents a problem in so far as these compounds are provided in the crystalline form and in so far as they are not or are only slightly soluble in water and in the fatty substances conventionally used in the cosmetic and dermatological fields. Thus, introduced as is into topical compositions, they do not dissolve and remain in the form of crystals, which may cause annoyance to or discomfort of the skin in use.

These derivatives can be partially dissolved in lower alcohols, such as ethanol or isopropanol, or solvents such as octyldodecanol, some glycols or short-chain fatty alcohols (lower than C₁₂). However, lower alcohols exhibit the disadvantage of drying out and of irritating the skin; it is therefore preferable to use them in reduced amounts, indeed even zero amounts, in products for the care of the body and/or face. In addition, these solubilizing agents can only be introduced in small amounts if a detrimental change in the cosmetic qualities (softness) and in the stability of the compositions comprising them is to be avoided.

The need thus remains to be able to dissolve imidopercarboxylic acid derivatives in the physiologically acceptable media of cosmetic and/or dermatological compositions.

### SUMMARY OF THE INVENTION

The inventors have found, unexpectedly, that lipophilic derivatives of amino acids in the form of esters make it possible to dissolve imidopercarboxylic acid derivatives without recrystallization of these derivatives occurring in media such as the physiologically acceptable media of cosmetic and dermatological compositions.

### DETAILED DESCRIPTION OF THE PREFERRED EMBODIMENTS

One subject of the present invention is a composition comprising, in a physiologically acceptable medium, at least one imidopercarboxylic acid compound and at least one ester chosen from N-acylated amino acid esters of formula (A):

R′₁(CO)N(R′₂)CH(R′₃)(CH₂)ₙ(CO)OR′₄ (A)

in which:

n is an integer equal to 0, 1 or 2,

R′₁₁ represents a linear or branched C₅ to C₂₁ alkyl or alkenyl radical,

R′₂ represents a hydrogen atom or a C₁ to C₃ alkyl group,

R′₃ represents a radical chosen from the group formed by a hydrogen atom, a methyl group, an ethyl group and a linear or branched C₃ or C₄ alkyl chain,

R′₄ represents a linear or branched C₁ to C₁₀ alkyl radical or a linear or branched C₂ to C₁₀ alkenyl radical or a sterol residue.

The term “physiologically acceptable medium” is understood to mean a medium compatible with keratinous substances, such as the skin, including the scalp, mucous membranes, eyes and hair.

The inventors of the present patent application have found that the use of lipophilic derivatives of amino acids in the form of esters makes it possible to unexpectedly increase the dissolution of the imidopercarboxylic acid derivatives described above and to obtain compositions which are stable (no formation of crystals) over time, which are also stable when the temperature is modified and which are satisfactory with regard to the cosmetic properties.

Another subject of the invention is a cosmetic treatment method which comprises applying the invention compositions to a keratinous substance.

Another subject of the invention is a method for the dissolution of an imidopercarboxylic acid derivative by an N-acylated amino acid ester of formula (A) as defined above.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

Imidopercarboxylic Acid Compound

The imidopercarboxylic acid derivative used in the composition according to the invention is preferably chosen from the compounds of following formula (I):

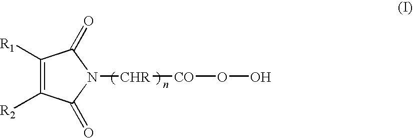

in which:
R₁ and R₂ represent, independently of one another, (i) a hydrogen atom, (ii) a (C₁-C₅)alkyl group optionally substituted by one or more, preferably by one to three, groups chosen from a (C₁-C₅)alkoxy group, the —OH group, the —COOH group, the —COOOH group, a —COOR₄ group, R₄ being as defined below, the —NO₂ group and a halogen atom or (iii) a (C₁-C₅)alkoxy group,
or else R₁ and R₂ together form
   either a 5- or 6-membered nonaromatic ring optionally substituted by one or more, preferably by one to three, groups chosen from a (C₁-C₅)alkyl group, optionally substituted by one or more, preferably by one to three, groups chosen from a (C₁-C₅)alkoxy group, the —OH group, the —COOH group, the —COOOH group, a —COOR₄ group, the —NO₂ group and a halogen atom, or a (C₁-C₅)alkoxy group and
R and n are as defined below,
   or an aromatic ring A, in order to more particularly form a compound which is an aromatic imidopercarboxylic acid derivative of formula (II),

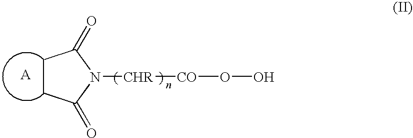

in which:
A represents a benzene or naphthalene ring optionally substituted by one or more, preferably by up to 4 and more preferably still by 1 or 2 groups chosen from an R₃ radical, a (C₁-C₅)alkoxy group, the —OH group, the —COOH group, the —COOOH group, a —COOR₄ group, the —NO₂ group and a halogen atom,
the R group or groups, which are identical or different, represent(s) a hydrogen atom, an —OH group, a —COOH group, a —COOOH group, a —COOR₄ group or an R₃ radical,
   R₃ represents a (C₁-C₅)alkyl group optionally substituted by one or more, preferably by one to three, groups chosen from a (C₁-C₅)alkoxy group, the —OH group, the —COOH group, the —COOOH group, a —COOR₄ group, the —NO₂ group and a halogen atom,
   R₄ represents a (C₁-C₅)alkyl group optionally substituted by a group chosen from a (C₁-C₅)alkoxy group, the —OH group, the —COOH group, the —COOOH group and a —COOR₅ group where R₅ is a (C₁-C₅)alkyl group, and
n is an integer varying from 1 to 5, for example from 1 to 3 or, for example, from 3 to 5.

It is understood that the compound of formula (I) or (II) comprises all the possible enantiomers and their mixtures and also the corresponding salts or any dimer of the compound of formula (I) or (II).

Mention may be made, among the salts, of the potassium salts or the ammonium salts, for example.

Examples of (C₁-C₅)alkyl groups are methyl, ethyl, n-propyl, isopropyl, n-butyl, isobutyl, sec-butyl, tert-butyl and pentyl. Mention may be made, among (C₁-C₅)alkoxy groups, of methoxy or ethoxy. Mention may be made, among halogen atoms, of fluorine, chlorine and bromine.

Mention is made in particular, among substituted (C₁-C₅)alkyl groups, of the —CH₂OCH₃, —CH₂OC₂H₅, —CH₂OH, —CH₂COOH, —CH₂COOOH, —CH₂CH₂COOH, —CH₂CH₂COOOH, —CH₂Cl, —CH₂F, —CF₃ and —CH₂COOC₂H₅ groups.

According to one embodiment, the R group is a hydrogen atom.

According to another embodiment, A represents a benzene ring.

According to yet another embodiment, A is substituted by at least one —COOH group or one —COOOH group.

Finally, according to another embodiment, A is unsubstituted.

According to one embodiment, use is made of an aromatic imidopercarboxylic acid derivative of formula (II) for which A is an unsubstituted benzene or naphthalene ring, R is a hydrogen atom, a —COOOH group or a (C₁-C₃)alkyl group optionally substituted by a —COOOH group or a —COOH group, and n varies from 3 to 5.

Mention may be made, among the compounds of formula (I), of the following compounds: 2,5-dihydro-3-methyl-2,5-dioxo-1H-pyrrole-1-hexaneperoxoic acid (R₁=H, R₂=Me; n=5), 2,5-dihydro-2,5-dioxo-1H-pyrrole-1-hexaneperoxoic acid (R₁=R₂=H; n=5), and 1,3,4,5,6,7-hexahydro-1,3-dioxo-2H-isoindole-2-butaneperoxoic acid (R₁ and R₂=6-membered ring; n=3).

Mention may be made, among the compounds of formula (II), as normal names, of the following compounds: phthalimidoperacetic acid (A=benzene; n=1), 3-phthalimidoperpropionic acid (A=benzene; n=2), 2-phthalimidodipersuccinic acid (A=benzene; R=COOOH; n=2), 3-phthalimidoperbutyric acid (A=benzene; R=CH₃; n=2), 2-phthalimidoperpropionic acid (A=benzene; R=CH₃; n=1), the methyl hemiester of 2-phthalimidomonoperglutaric acid (A=benzene; R=—COOOMe; n=3), 3-phthalimidodiperadipic acid (A=benzene; R=COOOH; n=4), naphthalimidoperacetic acid (A=naphthalene; n=1), 2-phthalimidomonopersuccinic acid (A=benzene; R=COOH; n=2) and 4-(4-percarboxyphthalimido)perbutyric acid.

Mention may also be made, among the compounds of formula (II), of the following compounds, cited as CAS names:
compounds for which A is a benzene ring, for example 1,3-dihydro-1,3-dioxo-2H-isoindole-2-butaneperoxoic acid, also known as phthalimidoperoxybutanoic acid, 3-phthalimidoperoxybutanoic acid or 4-phthalimidoperbutyric acid (n=3), 1,3-dihydro-1,3-dioxo-2H-isoindole-2-pentaneperoxoic acid (n=4), 1,3-dihydro-1,3-dioxo-2H-isoindole-2-heptaneperoxoic acid (n=6), 1,3-dihydro-1,3-dioxo-2H-isoindole-2-octaneperoxoic acid (n=7) or phthalimidoperoctanoic acid, 1,3-dihydro-1,3-dioxo-2H-isoindole-2-nonaneperoxoic acid (n=8), 1,3-dihydro-1,3-dioxo-2H-isoindole-2-decaneperoxoic acid (n=9), 1,3-dihydro-1,3-dioxo-2H-isoindole-2-undecaneperoxoic acid (n=10) or 1,3-dihydro-1,3-dioxo-2H-isoindole-2-dodecaneperoxoic acid (n=11);
compounds for which A is a substituted benzene ring, for example 1,3-dihydro-5-(hydroperoxycarbonyl)-1,3-dioxo-2H-isoindole-2-butaneperoxoic acid (n=3, substitution by a —COOOH group) or 1,3-dihydro-4-(hydroperoxycarbonyl)-1,3-dioxo-2H-isoindole-2-butaneperoxoic acid (n=3, substitution by a —COOOH group);
compounds for which A is a benzene ring and at least one of the R groups is other than a hydrogen atom, for example 3-(1,3-dihydro-1,3-dioxo-2H-isoindol-2-yl)hexanediperoxoic acid (n=3, R=CH₂COOOH), (1,3-dihydro-1,3-dioxo-2H-isoindol-2-yl)butanediperoxoic acid (R=COOOH, n=2), 2-(1,3-dihydro-1,3-dioxo-2H-isoindol-2-yl)pentanediperoxoic acid (R=COOOH, n=3), the 5-methyl ester of 4-phthalimidomonoperoxyglutaric acid (R=COOMe, n=3) or 1,3-dihydro-β-methyl-1,3-dioxo-2H-isoindole-2-propaneperoxoic acid, also known as β-methyl-1,3-dioxo-2-isoindolineperoxypropionic acid or 3-phthalimidoperbutyric acid (R=Me, n=2).

The following salt may also be mentioned: N-(2-hydroxyethyl)-N,N-dimethyl-1-dodecanaminium salt of 1,3-dihydro-1,3-dioxo-2H-isoindole-2-hexaneperoxoic acid (1:1).

According to yet another form, the compound of formula (II) is phthalimidoperoxycaproic acid, also known as 6-(phthalimido)peroxyhexanoic acid, ε-(phthalimido)peroxyhexanoic acid or PAP. Its chemical structure is as follows:

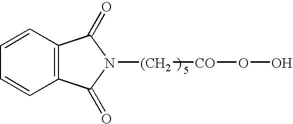

It corresponds to a compound of formula (II) in which the R group represents a hydrogen atom, A represents an unsubstituted benzene ring and n=5.

Finally, mention may be made, as acceptable dimer of a compound of formula (II), of the dimer of PAP, also known as 5,7-dihydro-1,3,5,7-tetraoxobenzo-[1,2-c:4,5-c′]dipyrrole-2,6(1H,3H)-dihexaneperoxoic acid of formula

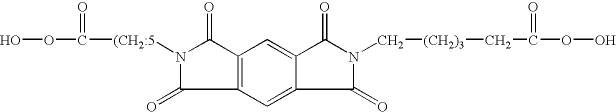

By way of example, the amount, as active materials, of imidopercarboxylic acid derivative, in particular of compound of formula (I) or (II), which can be used according to the invention can range, for example, from 0.01 to 5% by weight, in particular from 0.01 to 2% by weight, preferably from 0.15 to 1.5% by weight and better still from 0.3 to 1% by weight, with respect to the total weight of the composition.

Phthalimidoperoxycaproic acid or phthalimidoperoxyhexanoic acid (PAP) is available in particular in two commercial forms from Solvay.

Thus, phthalimidoperoxycaproic acid can be employed in conjunction with cyclodextrins, so as to improve the stability of the compound.

The document EP 895 777, already mentioned in the introduction, more particularly describes the formation of an adduct of ε-phthalimidoperoxyhexanoic acid with cyclodextrin.

Such an encapsulated product is sold by Solvay under the trade name Eureco® HC-P11 (Powdered inclusion complex of phthalimidoperoxycaproic acid with pharmagrade β-cyclodextrin).

Phthalimidoperoxycaproic acid is also supplied in the form of an aqueous dispersion (comprising 17% of active material) under the trade name Eureco® HC L17. Phthalimidoperoxycaproic acid is also supplied in the form of a powder under the trade name Eureco® HC-P11. Such aqueous dispersions of phthalimidoperoxycaproic acid are described in the document EP 1 074 607. These aqueous dispersions are in reality stabilized by copolymers of methyl vinyl ether with maleic acid and/or anhydride, in a 1:1 ratio, having an alternating structure.

Other formulation forms comprising an imidopercarboxylic acid derivative may also be suitable for an implementation of the present invention. Mention may in particular be made, as such, of dispersions, in particular aqueous dispersions, such as described in Patent Application WO 2004/110610, capsules comprising a gel-based matrix, such as described in Patent Application WO 2004/110611, capsules based on an inorganic salt, such as described in Application WO 2004/110612 or also multilayer capsules comprising at least one polyelectrolyte and/or one ionic surfactant, such as described in Patent Application WO 2004/110613.

N-Acylated Amino Acid Esters

The ester present in the composition according to the invention is advantageously chosen from N-acylated amino acid esters of formula:

R′₁(CO)N(R′₂)CH(R′₃)(CH₂)ₙ(CO)OR′₄

in which:

n is an integer equal to 0, 1 or 2,

R′₁ represents a linear or branched C₅ to C₂₁ alkyl or alkenyl radical,

R′₂ represents a hydrogen atom or a C₁ to C₃ alkyl group,

R′₃ represents a radical chosen from the group formed by a hydrogen atom, a methyl group, an ethyl group and a linear or branched C₃ or C₄ alkyl chain,

R′₄ represents a linear or branched C₁ to C₁₀ alkyl radical or a linear or branched C₂ to C₁₀ alkenyl radical or a sterol residue.

In the formula of the amino acid esters presented above, the R′₁(CO)— group is an acyl group of an acid preferably chosen from the group formed by capric acid, lauric acid, myristic acid, palmitic acid, stearic acid, behenic acid, linoleic acid, linolenic acid, oleic acid, isostearic acid, 2-ethylhexanoic acid, coconut oil fatty acids and palm kernel oil fatty acids. In addition, these fatty acids can exhibit a hydroxyl group. More preferably still, the acid will be lauric acid.

The —N(R′₂)CH(R′₃)(CH₂)ₙ(CO)— part of the amino acid ester is preferably chosen from the following amino acids: glycine, alanine, valine, leucine, isoleucine, serine, threonine, proline, hydroxyproline, β-alanine, aminobutyric acid, aminocaproic acid, sarcosine or N-methyl-β-alanine.

More preferably still, the amino acid will be sarcosine.

The part of the amino acid esters corresponding to the OR′₄ group can be obtained from alcohols chosen from the group formed by methanol, ethanol, propanol, isopropanol, butanol, tert-butanol, isobutanol, 3-methyl-1-butanol, 2-methyl-1-butanol, fusel oil, pentanol, hexanol, cyclohexanol, octanol, 2-ethylhexanol, decanol, lauryl alcohol, myristyl alcohol, cetyl alcohol, cetearyl alcohol, stearyl alcohol, oleyl alcohol, behenyl alcohol, jojoba alcohol, 2-hexadecylic alcohol, 2-octyldodecanol and isostearyl alcohol.

These amino acid esters can in particular be obtained from natural sources of amino acids. In this case, amino acids originate from the hydrolysis of natural plant proteins (oats, wheat, soya, palm, coconut) and then necessarily result in mixtures of amino acids which must subsequently be esterified and then N-acylated. The preparation of such amino acids is described more particularly in Patent Application FR 2 796 550, which is incorporated here by way of reference.

The N-acylated amino acid ester more particularly preferred for the use thereof in the present invention is isopropyl N-lauroylsarcosinate of formula:

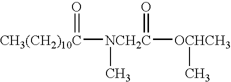

such as the product sold under the name Eldew SL-205 by Ajinomoto.

The N-acylated amino acid ester as described above can be present in the composition according to the invention in a content ranging for example from 0.05 to 50% by weight, preferably from 1 to 30% by weight and more preferably from 1 to 10% by weight, with respect to the total weight of the composition.

According to a preferred embodiment, the N-acylated amino acid ester and the imidopercarboxylic acid derivative of formula (I) are present in the composition according to the invention in contents as active materials such that the N-acylated amino acid ester to imidopercarboxylic acid derivative ratio ranges from 5 to 25 and better still from 10 to 20. Preferably, this ratio is based on weight.

The composition can also comprise a fatty phase which can comprise oils, gums or waxes normally used in the field of application under consideration. Mention may be made, as oils or waxes which can be used in the invention, of mineral oils (liquid petrolatum), vegetable oils (liquid fraction of shea butter, sunflower oil, apricot oil, rice bran oil, and the like), animal oils (perhydrosqualene), synthetic oils (Purcellin oil), silicone oils or waxes (cyclomethicone, dimethicone) and fluorinated oils (perfluoropolyethers), beeswax, carnauba wax, paraffin wax, shea butter or hydrogenated jojoba oil. To these oils can be added fatty alcohols (cetyl, stearyl, and the like) and fatty acids (stearic acid, and the like).

When a composition is an emulsion, the proportion of the fatty phase can range for example from 5% to 80% by weight and preferably from 5% to 50% by weight, with respect to the total weight of the composition. The oils, the waxes, the emulsifiers and the coemulsifiers used in the composition in the emulsion form can be chosen from those conventionally used in the cosmetics field. The emulsifier and the coemulsifier are preferably present in the composition in a proportion ranging from 0.3% to 30% by weight and more preferably from 0.5 to 20% by weight, with respect to the total weight of the composition. The emulsion can additionally comprise lipid vesicles.

When the composition is an oily solution or gel, the fatty phase can represent more than 90% of the total weight of the composition.

The composition can also comprise adjuvants usual in the field under consideration, such as surfactants, emulsifiers, hydrophilic or lipophilic gelling agents, hydrophilic or lipophilic additives, preservatives, antioxidants, solvents, fragrances, fillers, screening agents, odour absorbers, colouring materials or other cosmetic or pharmaceutical active principles. The amounts of these various adjuvants are those conventionally used in the cosmetics field, for example from 0.01% to 10% of the total weight of the composition. These adjuvants, depending on their nature, can be introduced into the fatty phase, into the aqueous phase and/or into the lipid spherules.

Mention may be made, as surfactants capable of being used, for example, of glycerol stearate, polysorbate 60, the PEG-6/PEG-32/glycol stearate mixture sold under the name Tefose® 63 by Gattefosse, PEG stearate derivatives or sugar derivatives.

Mention may be made, as hydrophilic gelling agents which can be used in the invention, of carboxyvinyl polymers (carbomer), acrylic copolymers, such as acrylate/alkyl acrylate copolymers, polyacrylamides, polysaccharides, such as hydroxypropylcellulose, natural gums and clays and mention may be made, as lipophilic gelling agents, of modified clays, such as bentones, metal salts of fatty acids, such as aluminium stearates, hydrophobic silica, ethylcellulose or polyethylene.

The composition can be provided in all the formulation forms which can be envisaged. In particular, the composition can have the form of an aqueous, alcoholic, aqueous/alcoholic or oily solution; of a dispersion of the lotion or serum type; of a water-in-oil, oil-in-water or multiple emulsion; of a suspension; of microcapsules or microparticles; of vesicular dispersions of ionic and/or non-ionic type; of an aqueous or oily lotion or of a lotion in the serum form; of a foam; of a solid preparation, for example a stick preparation; of an aerosol composition also comprising a pressurized propellant; or in the form of a patch.

The composition which can be used according to the invention can be provided in the form of a composition for haircare, in particular a shampoo, a hair setting lotion, a treating lotion, a styling cream or a styling gel, a dyeing composition, in particular an oxidation dyeing composition, hair restructuring lotions, a perming composition (in particular a composition for the first step of a permanent wave), a lotion or a gel for combating hair loss or an antiparasitic shampoo.

It can also be provided in the form of a cleaning, protecting, treating or care composition for the face, for the hands, for the feet, for the major anatomical folds or for the body (for example, day cream, night cream, make-up-removing cream, antisun composition, protective or care body milk, aftersun milks, lotion, gel or foam for caring for the skin, such as cleansing lotion, or artificial tanning composition); a composition for making up the body or face, such as a foundation; a bath composition; a deodorizing composition comprising, for example, a bactericidal agent; an aftershave composition; a depilatory composition; a composition for countering insect stings or bites; a pain-relieving composition; or a composition for treating certain skin diseases, such as eczema, rosacea, psoriasis, lichen or severe pruritus.

When the composition which can be used according to the invention is intended for a use of peeling type, it can also be provided in all the formulation forms mentioned above, provided that it is easily removed by rinsing, in particular in the form of an aqueous gel or an aqueous or aqueous/alcoholic solution. It can be applied by any means which makes possible uniform distribution, in particular using a cottonwool swab, a stick, a brush, a gauze, a spatula or a pad or also by spraying, and can be removed by rinsing with water, or using a mild detergent. According to a preferred embodiment of the invention, the composition intended for chemical peeling includes a continuous aqueous phase.

It can be provided in all the forms normally used for topical application, in particular in the form of an aqueous/alcoholic solution, of an oil-in-water or water-in-oil or multiple emulsion, of an oily gel or of a liquid, pasty or solid anhydrous product or in the form of a dispersion in the presence of spherules; these spherules can be polymeric nanoparticles, such as nanospheres or nanocapsules, or lipid vesicles of ionic or non-ionic type. These compositions are prepared according to the usual methods.

This composition can be more or less fluid and can have the appearance of a white or coloured cream, of an ointment, of a milk, of a lotion, of a serum, of a paste or of a foam. It can optionally be applied to the skin or hair in the aerosol form. It can also be provided in the solid form, for example in the stick form.

The following examples illustrate the present invention without implied limitation.

### Example 1

### Aqueous Gel

| | % by weight |
|---|---|
| Sodium hydroxide | 0.37 |
| Citric acid | 0.37 |
| Phthalimidoperoxycaproic acid, Eureco ® HCL17 (17% | 5 |
| by weight of active material) from Solvay | |
| Cetyl alcohol | 1 |
| Isopropyl N-lauroylsarcosinate (Eldew SL-205 from | 10 |
| Ajinomoto) | |
| Glyceryl stearate/polyethylene glycol (100 EO) | 0.6 |
| stearate mixture (Arlacel 165FL from Croda) | |
| Crosslinked acrylates/C₁₀-C₃₀ alkyl acrylate | 0.9 |
| polymer (Carbopol Ultrez-20 from Noveon) | |
| Polyethylene glycol (8 EO) | 6 |
| Preservative | 0.2 |
| Water | q.s. for 100 |

Procedure

The Carbopol is sprinkled into the aqueous phase at ambient temperature (25° C.) and left for 10 minutes in order for the powder to become impregnated with water. The combined mixture is stirred with a Rayneri mixer and then the sodium hydroxide is added while increasing the stirring. After a gel devoid of lumps has been obtained, the preservatives, the citric acid and the PEG-8 are added, followed by the PAP (homogenized beforehand with a bar for a few minutes). The stirring is maintained at a high level for a few minutes. At the same time, the fatty phase is prepared by heating the cetyl alcohol, the N-lauroylsarcosinate and the Arlacel at 50° C. and then the fatty phase is emulsified in the aqueous gel prepared above for 10 minutes with vigorous stirring.

### Example 2 and Comparative Examples 3 to 7

The following compositions have been prepared

| | | | | | |
|---|---|---|---|---|---|
| Example 2 | Example 3 | Example 4 | Example 5 | Example 6 | Example 7 |
| % by | % by | % by | % by | % by | % by |
| weight | weight | weight | weight | weight | weight |

Phase A

| | | | | | | |
|---|---|---|---|---|---|---|
| Sodium hydroxide | 0.2 | 0.2 | 0.2 | 0.2 | 0.2 | 0.2 |
| Disodium EDTA | 0.05 | 0.05 | 0.05 | 0.05 | 0.05 | 0.05 |
| Trisodium citrate | 0.05 | 0.05 | 0.05 | 0.05 | 0.05 | 0.05 |
| Zinc gluconate | 0.1 | 0.1 | 0.1 | 0.1 | 0.1 | 0.1 |
| Citric acid | 0.3 | 0.3 | 0.3 | 0.3 | 0.3 | 0.3 |
| Salicylic acid | 0.6 | 0.6 | 0.6 | 0.6 | 0.6 | 0.6 |
| Extract of | 0.02 | 0.02 | 0.02 | 0.02 | 0.02 | 0.02 |
| Eucalyptus | | | | | | |
| leaves | | | | | | |
| Xanthan gum | 0.2 | 0.2 | 0.2 | 0.2 | 0.2 | 0.2 |
| Glycerol | 5 | 5 | 5 | 5 | 5 | 5 |
| Preservatives | 1 | 1 | 1 | 1 | 1 | 1 |
| Water | Qsp100 | Qsp100 | Qsp100 | Qsp100 | Qsp100 | Qsp100 |

Phase B

| | | | | | | |
|---|---|---|---|---|---|---|
| 2-Octyldodecanol | 1 | 1 | 1 | 1 | 1 | 1 |
| 5-(n-Octanoyl)salicylic | 0.1 | 0.1 | 0.1 | 0.1 | 0.1 | 0.1 |
| acid | | | | | | |
| Polydimethyl- | 0.5 | 0.5 | 0.5 | 0.5 | 0.5 | 0.5 |
| siloxane 100 cSt | | | | | | |
| (DC 200 Fluid | | | | | | |
| from Dow Corning) | | | | | | |
| Cyclohexadimethyl- | 8.5 | 8.5 | 8.5 | 8.5 | 8.5 | 8.5 |
| siloxane, 8 cSt | | | | | | |
| (DC 246 Fluid | | | | | | |
| from Dow Corning) | | | | | | |
| Cetyl alcohol | 0.25 | 0.25 | 0.25 | 0.25 | 0.25 | 0.25 |
| Isopropyl N- | 7.5 | | | | | |
| lauroylsarcosinate | | | | | | |
| (Eldew SL-205 from | | | | | | |
| Ajinomoto) | | | | | | |
| Propylene carbonate | | 7.5 | | | | |
| Hexydecanol | | | 7.5 | | | |
| Octyl-2 dodecanol | | | | 7 | | |
| Branched C12-C13 | | | | | 7 | |
| alcool benzoates | | | | | | |
| (COSMACOL ELI | | | | | | |
| from Sasol) | | | | | | |
| Polyethylene | | | | | | 7.5 |
| glycol 400 (8 OE) | | | | | | |
| Glyceryl stearate/ | 1 | 1 | 1 | 1 | 1 | 1 |
| polyethylene glycol | | | | | | |
| (100 EO) | | | | | | |
| stearate mixture | | | | | | |
| (Arlacel 165FL | | | | | | |
| from Croda) | | | | | | |

Phase C

| | | | | | | |
|---|---|---|---|---|---|---|
| Crosslinked | 2 | 2 | 2 | 2 | 2 | 2 |
| polyacrylamidomethyl- | | | | | | |
| propanesulphonic acid | | | | | | |
| partially neutralized | | | | | | |
| with ammonia (Hostacerin | | | | | | |
| AMPS from Clariant) | | | | | | |

Phase D

| | | | | | | |
|---|---|---|---|---|---|---|
| Phthalimidoperoxy- | 2.5 | 2.5 | 2.5 | 2.5 | 2.5 | 2.5 |
| caproic acid, Eureco ® | | | | | | |
| HCL17 (17% by weight | | | | | | |
| as active material) | | | | | | |
| from Solvay | | | | | | |

Phase E

| | | | | | | |
|---|---|---|---|---|---|---|
| Ethyl alcohol | 5 | 5 | 5 | 5 | 5 | 5 |

Phase F

| | | | | | | |
|---|---|---|---|---|---|---|
| Polyamide fibres, | 3 | 3 | 3 | 3 | 3 | 3 |
| 0.9 dtex, length | | | | | | |
| 0.3 mm (Utexbel) | | | | | | |

Procedure

Phase A is heated with a portion of the water at 80° C. while stirring with a Rayneri mixer and then the mixture is cooled to 65° C. Phase B, heated beforehand on a water bath, is then added to phase A while stirring with the mixer (emulsification phase). The mixture, brought back to 45° C., is diluted with the remainder of the water and then phases C, D, E and F are successively added with stirring. Phase D can also be added during the diluting stage, before addition of phase C.

The aspect of the compositions has been evaluated:

| | | Example 3 | Example 4 | Example 5 | Example 6 |
|---|---|---|---|---|---|
| | Example 2 | com- | com- | com- | com- |
| | invention | parative | parative | parative | parative |
| Aspect | homogeneous | coarse | presence | very | very |
| | emulsions, | emulsion | of | coarse | coarse |
| | no crystals | | crystals, | emulsion, | emulsion, |
| | | | visible | presence | presence |
| | | | oily | of | of |
| | | | globules | crystals | crystals |

Contrarily to the other materials, only the N-acylated amino acid ester according to the invention allows a good solubilization of the phtalimidoperoxycaproic acid and gives an homogeneous emulsion, without crystals.

The above written description of the invention provides a manner and process of making and using it such that any person skilled in this art is enabled to make and use the same, this enablement being provided in particular for the subject matter of the appended claims, which make up a part of the original description and including a cosmetic or dermatological composition comprising, in a physiologically acceptable medium, at least one imidopercarboxylic acid derivative and at least one ester chosen from N-acylated amino acid esters of formula (A):

R′₁(CO)N(R′₂)CH(R′₃)(CH₂)ₙ(CO)OR′₄ (A)

in which:

n is an integer equal to 0, 1 or 2,

R′₁ represents a linear or branched C₅ to C₂₁ alkyl or alkenyl radical,

R′₂ represents a hydrogen atom or a C₁ to C₃ alkyl group,

R′₃ represents a radical chosen from the group formed by a hydrogen atom, a methyl group, an ethyl group and a linear or branched C₃ or C₄ alkyl chain,

R′₄ represents a linear or branched C₁ to C₁₀ alkyl radical or a linear or branched C₂ to C₁₀ alkenyl radical or a sterol residue.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in a physiologically acceptable medium, at least one imidopercarboxylic acid compound and at least one ester selected from the group consisting of N-acylated amino acid esters of formula (A):
R′₁(CO)N(R′₂)CH(R′₃)(CH₂)ₙ(CO)OR′₄ (A)
in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl chain,
R′₄ represents a linear or branched C₁ to C₁₀ alkyl radical or a linear or branched C₂ to C₁₀ alkenyl radical or a sterol residue.

2. The composition according to claim 1, in which the imidopercarboxylic acid compound is a compound of formula (I): 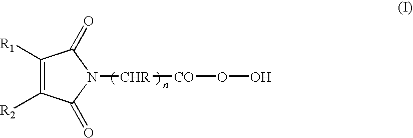
a salt thereof, or a dimer thereof,
in which:
R₁ and R₂ represent, independently of one another, (i) a hydrogen atom, (ii) a (C₁-C₅)alkyl group optionally substituted by one or more groups chosen from a (C₁-C₅)alkoxy group, an —OH group, a —COOH group, a —COOOH group, a —COOR₄ group, R₄ being as defined below, a —NO₂ group and a halogen atom or (iii) a (C₁-C₅)alkoxy group,
or else R₁ and R₂ together form
either a 5- or 6-membered nonaromatic ring optionally substituted by one or more groups chosen from a (C₁-C₅)alkyl group, optionally substituted by one or more groups chosen from a (C₁-C₅)alkoxy group, a —OH group, a —COOH group, a —COOOH group, a —COOR₄ group, a —NO₂ group and a halogen atom, or a (C₁-C₅)alkoxy group and
R and n are as defined below,
or an aromatic ring A, in order to more particularly form a compound which is an aromatic imidopercarboxylic acid derivative of formula (II), 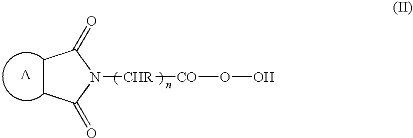
in which:
A represents a benzene or naphthalene ring optionally substituted by one or more groups chosen from an R₃ radical, a (C₁-C₅)alkoxy group, a —OH group, a —COOH group, a —COOOH group, a —COOR₄ group, a —NO₂ group and a halogen atom,
the R group or groups, which are identical or different, represent(s) a hydrogen atom, an —OH group, a —COOH group, a —COOOH group, a —COOR₄ group or an R₃ radical,
R₃ represents a (C₁-C₅)alkyl group optionally substituted by one or more groups chosen from a (C₁-C₅)alkoxy group, a —OH group, a —COOH group, a —COOOH group, a —COOR₄ group, a —NO₂ group and a halogen atom,
R₄ represents a (C₁-C₅)alkyl group optionally substituted by a group chosen from a (C₁-C₅)alkoxy group, a —OH group, a —COOH group, a —COOOH group and a —COOR₅ group where R₅ is a (C₁-C₅)alkyl group, and
n is an integer of 1 to 5.

3. The composition according to claim 2, in which R is a hydrogen atom.

4. The composition according to claim 2, in which A is a benzene ring.

5. The composition according to claim 1, in which the compound of formula (I) is at least one compound chosen from 2,5-dihydro-3-methyl-2,5-dioxo-1H-pyrrole-1-hexaneperoxoic acid, 2,5-dihydro-2,5-dioxo-1H-pyrrole-1-hexaneperoxoic acid and 1,3,4,5,6,7-hexahydro-1,3-dioxo-2H-isoindole-2-butaneperoxoic acid.

6. The composition according to claim 1, comprising at least one imidopercarboxylic acid compound chosen from phthalimidoperacetic acid, 3-phthalimidoperpropionic acid, 2-phthalimidodipersuccinic acid, 3-phthalimidoperbutyric acid, 2-phthalimidoperpropionic acid, the methyl hemiester of 2-phthalimidomonoperglutaric acid, 3-phthalimidodiperadipic acid, naphthalimidoperacetic acid, 2-phthalimidomonopersuccinic acid, 4-(4-percarboxyphthalimido)perbutyric acid, 1,3-dihydro-1,3-dioxo-2H-isoindole-2-butaneperoxoic acid, 3-phthalimidoperoxybutanoic acid, 1,3-dihydro-1,3-dioxo-2H-isoindole-2-pentaneperoxoic acid, 1,3-dihydro-1,3-dioxo-2H-isoindole-2-heptaneperoxoic acid, 1,3-dihydro-1,3-dioxo-2H-isoindole-2-octaneperoxoic acid, 1,3-dihydro-1,3-dioxo-2H-isoindole-2-nonane-peroxoic acid, 1,3-dihydro-1,3-dioxo-2H-isoindole-2-decaneperoxoic acid, 1,3-dihydro-1,3-dioxo-2H-isoindole-2-undecaneperoxoic acid, 1,3-dihydro-1,3-dioxo-2H-isoindole-2-dodecaneperoxoic acid, 1,3-dihydro-5-(hydroperoxycarbonyl)-1,3-dioxo-2H-isoindole-2-butaneperoxoic acid, 1,3-dihydro-4-(hydroperoxycarbonyl)-1,3-dioxo-2H-isoindole-2-butaneperoxoic acid, 3-(1,3-dihydro-1,3-dioxo-2H-isoindol-2-yl)hexanediperoxoic acid, (1,3-dihydro-1,3-dioxo-2H-isoindol-2-yl)butanediperoxoic acid, 2-(1,3-dihydro-1,3-dioxo-2H-isoindol-2-yl)pentanediperoxoic acid, the 5-methyl ester of 4-phthalimidomonoperoxyglutaric acid, 1,3-dihydro-β-methyl-1,3-dioxo-2H-isoindole-2-propaneperoxoic acid and the N-(2-hydroxyethyl)-N,N-dimethyl-1-dodecanaminium salt of 1,3-dihydro-1,3-dioxo-2H-isoindole-2-hexaneperoxoic acid (1:1).

7. The composition according to claim 1, comprising phthalimidoperoxycaproic acid.

8. The composition according to claim 1, in which the amount of the imidopercarboxylic acid compound is 0.01 to 5% by weight with respect to the total weight of the composition.

9. The composition according to claim 1, comprising isopropyl N-lauroylsarcosinate.

10. The composition according to claim 1, wherein said N-acylated amino acid ester is present in 0.05 to 50% by weight with respect to the total weight of the composition.

11. The composition according to claim 1, wherein the weight ratio of N-acylated amino acid ester to imidopercarboxylic acid compound is 5 to 25.

12. The composition according to claim 1, comprising 0.01 to 5% by weight with respect to the total weight of the composition of phthalimidoperoxycaproic acid and 0.05 to 50% by weight with respect to the total weight of the composition isopropyl N-lauroylsarcosinate.

13. A method, comprising applying a composition according to claim 1 to a keratinous substance.

14. The method according to claim 13, wherein the keratinous substance is the skin of a human.

15. A method for the dissolution of an imidopercarboxylic acid compound by an N-acylated amino acid ester of formula (A):
R′₁(CO)N(R′₂)CH(R′₃)(CH₂)ₙ(CO)OR′₄ (A)
in which:
n is an integer equal to 0, 1 or 2,
R′₁ represents a linear or branched C₅ to C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl chain,
R′₄ represents a linear or branched C₁ to C₁₀ alkyl radical or a linear or branched C₂ to C₁₀ alkenyl radical or a sterol residue,
said method comprising mixing said imidopercarboxylic acid compound and said N-acylated amino acid ester of formula (A) in a physiologically acceptable medium in a weight ratio of N-acylated amino acid ester to imidopercarboxylic acid compound of 5 to 25.
